(19) United States
(12) Patent Application Publication
(10) Pub. No.: US 2016/0324872 A1
(43) Pub. Date: Nov. 10, 2016

(54) IMPREGNATED DRESSINGS FOR TREATING DERMATOLOGICAL DISORDERS

(71) Applicant: MAYO FOUNDATION FOR MEDICAL EDUCATION AND RESEARCH, Rochester, MN (US)

(72) Inventors: Alison J. Bruce, Rochester, MN (US); Charles J. Bruce, Rochester, MN (US); Benjamin J. Barrick, Rochester, MN (US)

(73) Assignee: Mayo Foundation for Medical Education and Research, Rochester, MN (US)

(21) Appl. No.: 15/110,559

(22) PCT Filed: Jan. 8, 2015
(86) PCT No.: PCT/US15/10582

§ 371 Date: Jul. 8, 2016

Related U.S. Application Data
(60) Provisional application No. 61/924,816, filed on Jan. 8, 2014.

(51) Int. Cl. A61K 31/56; A61K 47/06; A61K 31/351; A41D 13/12; A61F 13/08; A41D 1/04; A41D 13/11; A41D 1/06; A61K 9/00; A41D 19/00

(52) U.S. Cl. CPC A61K 31/56; A61K 9/0087; A61K 47/06; A61K 31/351; A61K 9/0014; A41D 19/00; A61F 13/08; A41D 1/04; A41D 13/11; A41D 1/06; A41D 13/1263; A41D 2400/32

(57) ABSTRACT

This document provides methods and materials for treating dermatologic disorders. For example, methods and materials for using dressings (e.g., wet dressings) to treat dermatologic conditions such as dermatitis are provided.

### CROSS-REFERENCE TO RELATED APPLICATIONS

This application claims the benefit of U.S. Provisional Application Ser. No. 61/924,816, filed Jan. 8, 2014. The disclosure of the prior application is considered part of (and is incorporated by reference in) the disclosure of this application.

### TECHNICAL FIELD

This document relates to methods and materials for treating dermatologic disorders. For example, this document provides methods and materials for using dressings (e.g., wet dressings) to treat dermatologic conditions such as dermatitis.

### BACKGROUND

Management of dermatitis, particularly atopic dermatitis and irritant hand dermatitis, relies heavily on topical care using corticosteroids and wet dressings. Wet dressing therapy can be difficult to provide on an outpatient basis, however, because it is labor intensive—requiring the application of topical creams to the skin, wrapping with soaked gauze dressings or towels, and then covering with a dry, soft cloth or article of clothing. In addition, patients must be treated regularly (e.g., twice daily) to manage dermatitis flares.

### SUMMARY

This document is based, at least in part, on the development of dressings (e.g., wet dressings) that can be used by patients at home, or can be applied in an outpatient setting with minimal nursing intervention. The dressings can include, for example, articles of clothing that contain or are made from a material impregnated with one or more agents (e.g., dehydrated, topical steroids) that can be activated upon contact with water. In some cases, an article of manufacture (e.g., clothing) can be produced to include material impregnated with one or more active agents (e.g., active topical steroids) that can be active without requiring contact with water.

In one aspect, this document features an article of manufacture that includes a material coated or impregnated with one or more dehydrated steroid compounds, wherein the article is configured for placement on a human body part. The article can be, for example, a glove, a sock, a vest, a face mask, pants, or a whole-body suit. The entire article can be made from the material, or a portion of the article can be made from the material. The material can be a nylon or gauze fabric. The one or more steroids can include triamcinolone or hydrocortisone.

In another aspect, this document features a method for treating a dermatological condition on an affected body part. The method can include applying to the body part an article of manufacture including a material coated or impregnated with one or more dehydrated steroid compounds, wherein the article is configured for placement on the affected body part; and contacting the material with a sufficient amount water to activate the one or more steroid compounds. The contacting step can be carried out before the applying step, or the contacting step can be carried out after the applying step. The contacting step can include applying water to the surface of the material, and/or immersing the material in water. The article can be, for example, a glove, a sock, a vest, a face mask, pants, or a whole-body suit. The entire article can be made from the material, or a portion of the article can be made from the material. The material can be a nylon or gauze fabric. The one or more steroids can include triamcinolone and/or hydrocortisone.

In another aspect, this document features an article of manufacture comprising a material coated or impregnated with (a) one or more steroid compounds and (b) petrolatum or an antibiotic, wherein the article is configured for placement on a human body part. The article can be a glove, a sock, a vest, a face mask, pants, or a whole-body suit. The entire article can be made from the material. A portion of the article can be made from the material. The material can comprise a nylon or gauze fabric. The material can be coated or impregnated with triamcinolone or hydrocortisone. The material can be coated or impregnated with petrolatum. The material can be coated or impregnated with mupirocin.

In another aspect, this document features a method for treating a dermatological condition on an affected body part, comprising applying to the body part an article of manufacture. The article of manufacture can comprise a material coated or impregnated with (a) one or more steroid compounds and (b) petrolatum or an antibiotic, wherein the article is configured for placement on a human body part. The article can be a glove, a sock, a vest, a face mask, pants, or a whole-body suit. The entire article can be made from the material. A portion of the article can be made from the material. The material can comprise a nylon or gauze fabric. The material can be coated or impregnated with triamcinolone or hydrocortisone. The material can be coated or impregnated with petrolatum. The material can be coated or impregnated with mupirocin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

Wet dressing treatment is widely used for dermatological disorders such as dermatitis, including atopic dermatitis and irritant hand dermatitis (as may occur in, e.g., health care workers, factory workers, and domestic workers). In some cases, such therapies can be difficult to provide on an outpatient basis.

This document is based, at least in part, on the development of dressings (e.g., wet dressings) that can be used by patients at home, or that can be applied in an outpatient setting with minimal nursing intervention. For example, this document provides articles of manufacture including such dressings, as well as methods for making and using the articles of manufacture.

In some embodiments, a dressing provided herein can include an article of clothing that contains or is made from a material impregnated with one or more dehydrated, topical steroids or corticosteroids (e.g., hydrocortisone-type compounds or acetonide-type compounds such as triamcinolone), immunomodulatory agents (e.g., tacrolimus or pimecrolimus), moisturizers/emollients, and/or other ingredients (e.g., acetic acid and/or an antibiotic such as mupirocin) that can be activated upon contact with water. Such steroids can be included in amounts ranging from about 0.01% to about 5% (e.g., about 0.01%, about 0.025%, about 0.05%, about 0.075%, about 0.1%, about 0.25%, about 0.5%, about 0.75%, about 1.0%, about 1.25%, about 1.5%, about 1.75%, about 2.0%, about 2.25%, about 2.5%, about 2.75%, about 3.0%, about 3.5%, about 4.0%, about 4.5%, or about 5%). In some cases, an article of manufacture provided herein (e.g., an article of clothing) can include one or more dehydrated, topical corticosteroids (e.g., hydrocortisone-type compounds or acetonide-type compounds such as triamcinolone), immunomodulatory agents (e.g., tacrolimus or pimecrolimus), or moisturizers/emollients in an amount that ranges from about 0.1 percent to about 5 percent (e.g., from about 0.5 percent to about 5 percent, from about 1 percent to about 5 percent, from about 0.1 percent to about 2.5 percent, from about 0.1 percent to about 1 percent, or from about 0.5 percent to about 2.5 percent). For example, an article of manufacture provided herein can contain about 0.1% triamcinolone and/or about 2.5% hydrocortisone.

In some embodiments, a dressing provided herein can include an article of clothing that contains or is made from a material impregnated with one or more topical steroids or corticosteroids (e.g., hydrocortisone-type compounds or acetonide-type compounds such as triamcinolone), immunomodulatory agents (e.g., tacrolimus or pimecrolimus), moisturizers/emollients, and/or other ingredients (e.g., acetic acid and/or an antibiotic such as mupirocin) that are active without requiring contact with water. For example, a dressing provided herein can include an article of clothing that contains or is made from a material impregnated with one or more topical corticosteroids and one or more antibiotics (e.g., mupirocin). A steroid can be included in amounts ranging from about 0.01% to about 5% (e.g., about 0.01%, about 0.025%, about 0.05%, about 0.075%, about 0.1%, about 0.25%, about 0.5%, about 0.75%, about 1.0%, about 1.25%, about 1.5%, about 1.75%, about 2.0%, about 2.25%, about 2.5%, about 2.75%, about 3.0%, about 3.5%, about 4.0%, about 4.5%, or about 5%). In some cases, an article of manufacture provided herein (e.g., an article of clothing) can include one or more topical steroids or corticosteroids (e.g., hydrocortisone-type compounds or acetonide-type compounds such as triamcinolone), immunomodulatory agents (e.g., tacrolimus or pimecrolimus), moisturizers/emollients, and/or other ingredients (e.g., acetic acid and/or an antibiotic such as mupirocin) in an amount that ranges from about 0.1 percent to about 5 percent (e.g., from about 0.5 percent to about 5 percent, from about 1 percent to about 5 percent, from about 0.1 percent to about 2.5 percent, from about 0.1 percent to about 1 percent, or from about 0.5 percent to about 2.5 percent). For example, an article of manufacture provided herein can contain about 0.1% triamcinolone and/or about 2.5% hydrocortisone, both of which are active without requiring contact with water.

Examples of steroids that can be used as described herein include, without limitation, flurandrenolide, mometasone furoate, triamcinolone acetonide, fluocinolone acetonide, desoximetasone, fluocinolone acetonide, flurandrenolide, fluticasone propionate, prednicarbate, desonide, hydrocortisone, fluocinolone acetonide, alclometasone dipropionate, and clobetasol.

Suitable articles of clothing include, without limitation, gloves, socks, face masks, vests, pants, and whole-body suits. The articles can contain or be made from one or more materials that are disposable and/or inexpensive. For example, the articles can contain or be made from a nylon fabric (e.g., a nylon hose-type fabric), a gauze fabric (e.g., paper-like gauze), cotton, and other absorbent materials.

During the manufacturing process, the articles can be coated or impregnated with the one or more dehydrated (e.g., powdered) topical steroids, one or more active topical steroids (e.g., a topical steroid that is active without requiring contact with water), or a combination of both (a) one or more dehydrated (e.g., powdered) topical steroids and (b) one or more active topical steroids. In some cases, the article of manufacture provided herein can be coated or impregnated with other ingredients. For example, an article of manufacture provided herein (e.g., an article of clothing containing one or more dehydrated, topical steroids or an article of clothing containing one or more active topical steroids) can include white petrolatum. In some cases, white petrolatum can be infused into an article of manufacture provided herein for emollition purposes.

In some cases, an article of manufacture (e.g., an article of clothing) can be made to include white petrolatum while lacking steroids (e.g., while lacking topical steroids). For example, an article of manufacture provided herein can include white petrolatum and no steroid compounds.

Any appropriate amount of white petrolatum can be applied to an article of manufacture provided herein. For example, a thin layer of white petrolatum can be added to an article of manufacture provided herein to create an article of manufacture having both a steroid and white petrolatum.

Any appropriate method can be used to compound such steroids and other ingredients (e.g., acetic acid and/or an antibiotic such as mupirocin) and to make an article of manufacture provided herein. For example, a steroid and/or petrolatum infused product (e.g., sleeves, gloves, shirts, pants, facemasks, or ear covers) can be produced using materials or fabrics such as cotton or gauze. In some cases, a scaffold can be used when infusing a material with a steroid and/or petrolatum. For example, a petrolatum-based material (e.g., ointment or medication) or steroid-containing material can be gently heated to decrease viscosity as to not change the molecular structure of the material. White petrolatum can have a melting point of about 36° C. to about 60° C. Hydrocortisone can have a melting point of about 217° C. and about 220° C. The fabric-based product (e.g., a garment, gloves, or sleeve) can be rolled over a scaffold and placed in a pan. For example, an elasticated tubular bandage for use as a sleeve can be applied over a cylinder of similar length. The heated petrolatum-based material and/or steroid-containing material can be poured over the fabric-based product and underlying scaffold such that, for example, the fabric is coated thoroughly. Then, the product can be allowed to cool to room temperature. Once cooled, the infused product can be rolled off the scaffold and packaged or stored in a sealed package (e.g., a sealed bag) or container in a rolled form.

In some cases, an article of manufacture provided herein can be produced by soaking a fabric-based product (e.g., a garment, gloves, or sleeve) in steroid-containing solution. In some cases, the fabric-based product can be stored wet until use. In some cases, the fabric-based product can be allowed to dry and stored in a dry form.

When using an article with one or more dehydrated steroids as described herein, the user can apply the articles (e.g., gloves, socks, etc.) having dehydrated steroid(s) to the affected body surface(s), and can activate the dehydrated steroid(s) by contact with water. The contacting step can include, for example, applying water to the surface of the article, or immersing the article in water; the contacting step also can be carried out before or after the article is placed on the affected body surface. Thus, this document provides methods for using the articles provided herein. The methods can include placing an article impregnated with one or more dehydrated, topical steroids on an affected body surface, and then contacting the article with a sufficient amount of water to activate the steroid(s). A “sufficient” amount can be, for example, an amount obtained by dampening the article with water, or an amount obtained by immersing the article in water. The act of wetting the article also can bring the article into closer contact with the affected body surface. The methods can be quick and easy for patients to use in an outpatient setting, removing or reducing the need for skilled nursing.

In some cases, a pair of gloves can be designed as work gloves for users with occupational related hand dermatitis. For example, gloves can be designed to have an exterior layer (e.g., a hypoallergenic, water proof exterior layer) and an inner layer (e.g., a thin inner cotton layer). In these cases, the inner layer can include one or more dehydrated steroids, one or more active steroids, and/or white petrolatum. In some cases, the user can apply lotion, cream, or ointment (e.g., medicated or non-medicated lotion, cream, or ointment) underneath the glove. In some cases, a work glove can be amenable to washing for reuse or can be disposable.

In some cases, a dressing can be designed in a rolled “bi-layer tube” configuration suitable for application to extremities (or trunk), with two layers. In some cases, an inner layer can be a steroid impregnated absorbent layer, moistened with water, and an outer layer rolling over the inner layer as the dry occlusive layer.

In some cases, an article of manufacture provided herein can be provided in a rolled configuration. For example, a glove or shirt containing a steroid and/or white petrolatum can be packaged in a rolled configuration to allow a user to remove the glove or shirt from the package and apply the glove or shirt easily.

In some cases, a user can obtain an article of manufacture provided herein in a sealed container or package. The user can open that container or package and apply the product to the intended body area. For example, users with hand dermatitis can slide their fingers into rolled gloves and roll the remainder of the glove over their wrist and forearm. After a period of time, the user can remove and discard the product.

In some cases, the articles of manufacture provided herein can be used to treat dermatitis in an inexpensive, safe, and effective manner that is widely available to all patients regardless of prior expertise in administering dressings (e.g., wet dressings). For example, patients with hand dermatitis can use petrolatum-containing gloves provided herein underneath a second pair of gloves at night for soothing of dry, fissured, and/or dermatitic skin. In some cases, patients with occupation-related hand dermatitis (e.g., hair dressers) can use petrolatum-containing gloves provided herein under working gloves while performing their work duties. In some cases, a child with an atopic dermatitis flare could be treated proactively as described herein by parents, for example, when standard topical therapy is ineffective.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An article of manufacture comprising a material coated or impregnated with one or more dehydrated steroid compounds, wherein the article is configured for placement on a human body part.

2. The article of manufacture of claim 1, wherein the article is a glove, a sock, a vest, a face mask, pants, or a whole-body suit.

3. The article of manufacture of claim 1, wherein the entire article is made from the material.

4. The article of manufacture of claim 1, wherein a portion of the article is made from the material.

5. The article of manufacture of claim 1, wherein the material comprises cotton, a nylon, or a gauze fabric.

6. The article of manufacture of claim 1, wherein said material is coated or impregnated with triamcinolone, hydrocortisone, flurandrenolide, mometasone furoate, triamcinolone acetonide, fluocinolone acetonide, desoximetasone, fluocinolone acetonide, flurandrenolide, fluticasone propionate, prednicarbate, desonide, hydrocortisone, fluocinolone acetonide, alclometasone dipropionate, or clobetasol.

7. A method for treating a dermatological condition on an affected body part, comprising:
applying to the body part an article of manufacture comprising a material coated or impregnated with one or more dehydrated steroid compounds, wherein the article is configured for placement on the affected body part; and
contacting the material with a sufficient amount water to activate the one or more steroid compounds.

8. The method of claim 7, wherein the contacting step is carried out before the applying step.

9. The method of claim 7, wherein the contacting step is carried out after the applying step.

10. The method of claim 7, wherein the contacting step comprises applying water to the surface of the material.

11. The method of claim 7, wherein the contacting step comprises immersing the material in water.

12. The method of claim 7, wherein the article is a glove, a sock, a vest, a face mask, pants, or a whole-body suit.

13. The method of claim 7, wherein the entire article is made from the material.

14. The method of claim 7, wherein a portion of the article is made from the material.

15. The method of claim 7, wherein the material comprises cotton, a nylon, or gauze fabric.

16. The method of claim 7, wherein the one or more steroids is triamcinolone, hydrocortisone, flurandrenolide, mometasone furoate, triamcinolone acetonide, fluocinolone acetonide, desoximetasone, fluocinolone acetonide, flurandrenolide, fluticasone propionate, prednicarbate, desonide, hydrocortisone, fluocinolone acetonide, alclometasone dipropionate, or clobetasol.

17. An article of manufacture comprising a material coated or impregnated with (a) one or more steroid compounds and (b) petrolatum or an antibiotic, wherein the article is configured for placement on a human body part.

18. The article of manufacture of claim 17, wherein the article is a glove, a sock, a vest, a face mask, pants, or a whole-body suit.

19. The article of manufacture of claim 17, wherein the entire article is made from the material.

20. The article of manufacture of claim 17, wherein a portion of the article is made from the material.

21. The article of manufacture of claim 17, wherein the material comprises cotton, a nylon, or gauze fabric.

22. The article of manufacture of claim 17, wherein the material is coated or impregnated with triamcinolone, hydrocortisone, flurandrenolide, mometasone furoate, triamcinolone acetonide, fluocinolone acetonide, desoximetasone, fluocinolone acetonide, flurandrenolide, fluticasone propionate, prednicarbate, desonide, hydrocortisone, fluocinolone acetonide, alclometasone dipropionate, or clobetasol.

23. The article of manufacture of claim 17, wherein the material is coated or impregnated with petrolatum.

24. The article of manufacture of claim 17, wherein the material is coated or impregnated with mupirocin.

25. A method for treating a dermatological condition on an affected body part, comprising applying to the body part an article of manufacture comprising a material coated or impregnated with (a) one or more steroid compounds and (b) petrolatum or an antibiotic, wherein the article is configured for placement on a human body part.
